(19) United States
(12) Patent Application Publication
(10) Pub. No.: US 2011/0142861 A1
(43) Pub. Date: Jun. 16, 2011

(54) DEPLETION OF CD103 EXPRESSING CELLS FOR TREATMENT OF DISORDERS

(76) Inventor: Gregg Allen Hadley, New Albany, OH (US)

(21) Appl. No.: 11/848,230

(22) Filed: Aug. 30, 2007

Related U.S. Application Data
(60) Provisional application No. 60/824,007, filed on Aug. 30, 2006.

(51) Int. Cl. A61K 39/395; C07K 16/28; C07K 14/415; C07K 14/42; A61P 37/06

(52) U.S. Cl. 424/183.1; 424/178.1; 530/391.7; 530/370; 530/377

(57) ABSTRACT

The present invention relates to methods of selectively depleting CD103+ cells in vivo in a subject, with the methods comprising administering a composition comprising an anti-CD103 antibody conjugated to a lethal compound to the subject.

### CROSS-REFERENCE TO RELATED APPLICATIONS

This application claims the benefit of U.S. Provisional Application No. 60/824,007, filed Aug. 30, 2006, the entirety of which is hereby incorporated by reference.

### STATEMENT REGARDING FEDERALLY SPONSORED RESEARCH OR DEVELOPMENT

Part of the work performed during development of this invention utilized U.S. Government funds through NIH Grant No. RO1 AI-36532. The U.S. Government has certain rights in this invention.

### BACKGROUND OF THE INVENTION

1. Field of the Invention

The present invention relates to methods of selectively depleting CD103+ cells in vivo in a subject, with the methods comprising administering a composition comprising an anti-CD103 antibody conjugated to a lethal compound to the subject.

2. Background of the Invention

Graft-versus-host disease (GVHD) remains the primary complication of clinical bone marrow transplantation (BMT) and a major impediment to widespread application of this important therapeutic modality. The hallmark of GVHD is infiltration of donor T lymphocytes into host epithelial compartments of the skin, intestine, and biliary tract. GVHD occurs when mature T cells, contained in the bone marrow of the graft, are transplanted into immuno-suppressed hosts. After transplantation, host antigen presenting cells (APCs) activate T cells of the graft (donor T cells) by presenting host histocompatibility antigens to the graft T-cells. Donor-derived APCs may also activate donor T cells by cross-presenting host alloantigens. The newly generated host-specific T effector (hsTeff) populations then migrate to peripheral host organs and effect target organ damage.

Intestinal injury is one of the earliest and most common features of GVHD. T cell infiltration into the intestinal epithelium not only is requisite for intestinal pathology during GVHD, but also profoundly impacts the severity and overall mortality of GVHD (10). Several lines of evidence point to a key role for host-specific CD8+ T effector populations (hsCD8eff) in the development and progression of GVHD. In experimental GVHD models, hsDB8eff cells are associated with the most severe forms of intestinal pathology and predominate at the site of intestinal injury. Selective depletion of CD8+ T cells reduces the incidence of GVHD following BMT. Depletion of CD4+ cells, on the other hand, has no effect on the incidence of GVHD.

Attention to reducing the incidence of GVHD previously focused on recognition of tissue-specific major histocompatibility complex I (MHC I)/peptide complexes (See Steinmuller, D., Immunology Today 5:234-240 (1984)). However, recent studies indicate that the gut tropism of hsTeff populations is determined, in large part, by the pattern of adhesion molecules expressed on the cell surface of the intestinal epithelium.

The CD103/β7 (herein referred to as CD103) integrin, which can be expressed on T cells, has been shown to play a critical role in targeting epithelial allografts for destruction by CD8 effectors populations (Feng, Y., D., et al., J Exp Med 196:877-886 (2002), incorporated by reference). To date, however, it has not been shown that CD103 plays a significant role in directing hsTeff-induced GVHD.

Graft-derived T cells are, however, still necessary to treat tumors and other infections in immunosuppressed patients. Thus, ablating all graft derived T cells is not considered an option for treating GVHD. Thus what is needed in the art are methods of treating or preventing T cell-mediated GVHD while still retaining the benefits and protection that graft-derived T cells confer upon the host. Such methods may exploit the fact that GVHD may be, at least in part, mediated by tissue specific expression of cell adhesion molecules, rather than MHC/peptide complexes.

### SUMMARY OF THE INVENTION

The present invention relates to methods of selectively depleting CD103+ cells in vivo in a subject, with the methods comprising administering a composition comprising an anti-CD103 antibody conjugated to a lethal compound to the subject.

The present invention also relates to compositions comprising an anti-CD103 antibody conjugated to a lethal compound. In one embodiment, the lethal compound conjugated to the anti-CD103 antibody is a saporin protein.

### BRIEF DESCRIPTION OF THE DRAWINGS

FIG. 1 depicts flow cytometry data identifying cell populations using cell surface markers CD8 and CD44 in various organs in a mouse model of GVHD. Data shown on the left are dot plots of 1B2 vs. CD8 expression in lymphocyte populations. Data on the right are histograms of CD44 expression in gated 1B2+CD8+ lymphocytes. Lymphocytes were isolated from the various host compartments at 7-days post bone marrow transplant.

FIG. 2 depicts flow cytometry data showing expression of CD103 in hsCD8eff cells in the host gut in a mouse model of GVHD. Data shown are histograms of CD103 expression by gated host-specific CD8⁺ T cells (1B2⁺CD8⁺) lymphocytes. Isotype control staining is represented by shaded histograms. Numbers in top panel show the mean percentage (+SE) of intestinal CD8⁺ T cells that expressed CD103.

FIG. 3 depicts flow cytometry data identifying characteristics of both wild-type hsCD8eff cells and CD103 knockout hsCD8eff cells that infiltrate the gut in a mouse model of GVHD. Data shown are histograms showing expression of CD11a (A), CD62L (B), TNF-α (C), IFN-γ (D), and Annexin-V (E) by wild type (left) or CD103^{-/-} (right) host-specific CD8 cells (gated Thy1.1⁺1B2⁺ or Thy1.1⁻1B2⁺ cells, respectively) isolated from the intestinal epithelium at day 6. The shaded histograms represent isotype control staining (F) Data shown are CSFE (carboxyfluorescein diacetate succinimidyl ester) fluorescence by wild type (left) or CD103^{-/-} (right) host-specific CD8 cells (gated Thy1.1⁺1B2⁺ or Thy1.1⁻1B2⁺ cells, respectively) isolated from the intestinal epithelium. Filled histograms show CSFE fluorescence at day 0.

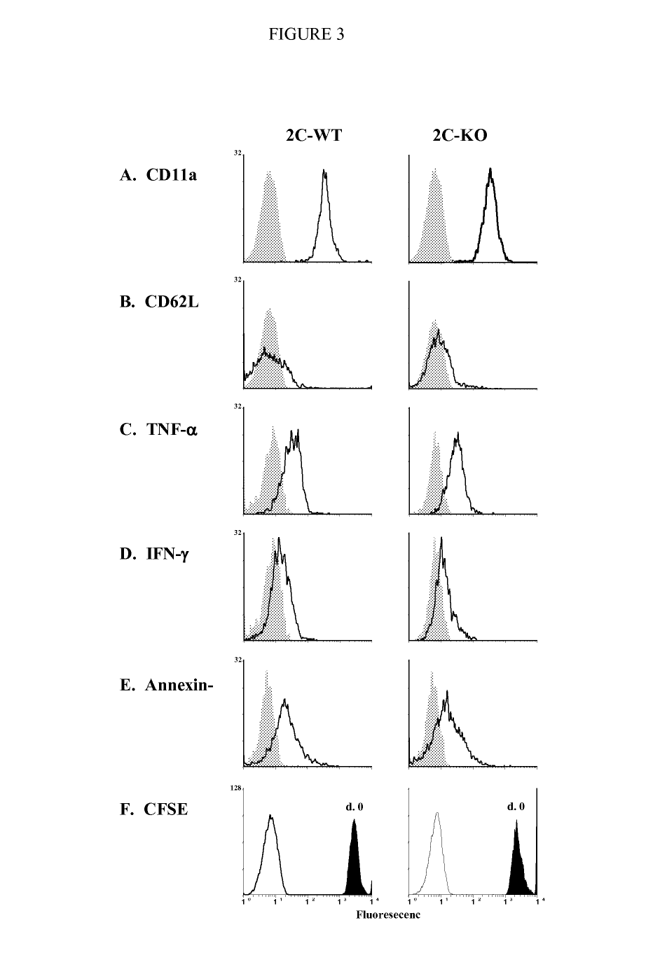

FIG. 4 depicts flow cytometry data showing that expression of CD103 is correlated with retention of hsCD8eff cells in the gut in a mouse model of GVHD. (A) Data shown are dot plots of Thy1.1 vs. 1B2 expression by gated CD8⁺ lymphocytes isolated from the intestinal epithelium (upper) or spleen (lower) at the indicated time points. (B) Data shown are dot plots of Thy1.1 vs. 1B2 expression by gated CD8⁺ lymphocytes isolated from the indicated organs at day 28 post-BMT. Numbers in quadrants denote the corresponding percentages among gated CD8⁺ T cells.

FIG. 5 depicts flow cytometry data showing expression of CD103 in polyclonal CD8+ cells in the host gut in a mouse model of GVHD. Data shown are dot plots of CD103 vs. CD8 expression by gated donor (H-2K^{b+}) lymphocytes infiltrating the host intestinal epithelium.

FIG. 6 depicts a tumor metastasis incidence curve showing the incidence of detectable metastases in mice receiving CD8+ cells. The curve shows a percentage of mice without detectable metastases. In mice receiving CD103 deficient cells and wild-type CD8+ cells, no metastases were detectable at 10 weeks, post transplantation.

FIG. 7 depicts flow cytometry data showing depletion of CD8+ T cells after treatment of mice with anti-CD103 immunotoxin. Data shown are density plots of CD8 expression vs. CD103 expression in gated lymphocytes.

FIG. 8 depicts the results of experiments wherein pancreatic islet allografts from BALB/c (H-2d) donors were transplanted k.c. into fully allogeneic C57BL/6 (H-2^{b}) recipients rendered diabetic by standard treatment with streptotocin. Recipients were untreated (open symbols and dashed line, n=7) or treated with 2 mg/kg i.p. of M290-saporin conjugate (M290-SAP) at day 3 post-transplant (solid symbols and solid line, n=8). Blood glucose (BG) was measured at biweekly intervals until the time of rejection (BG>200 mg/dl). Data shown are BG values accrued over time, with each line representing BG values obtained for a given recipient.

### DETAILED DESCRIPTION OF THE INVENTION

The present invention relates to methods of selectively depleting CD103+ cells in vivo in a subject, with the methods comprising administering a composition comprising an anti-CD103 antibody conjugated to a lethal compound to the subject. As used herein, the term “host subject” is intended to mean an animal that is receiving or has received a graft in the form of a bone marrow transplant (BMT). In one embodiment, the subject is a mammal. In a more specific embodiment, the host subject is a non-human or human primate.

The graft material of the present invention may be an isograft in relation to the host, an allograft in relation to the host or even a xenograft in relation to the host. The term isograft is used as it is in art to mean a graft taken from an organism that is genetically identical to the host subject, such as an identical twin or cloned organism. The term allograft is used as it is in the art to mean a graft taken from an organism that is a member of the same species as the host subject, for example a human graft to a non-genetically identical human. The term xenograft is used as it is in the art to mean a graft from an organism that is not a member of the same species as the host subject, for example a porcine graft to a human host. GVHD is associated with bone marrow transplant procedures and generally occurs in an acute and chronic form. Acute GVHD will be observed within about the first 100 days post BMT, whereas chronic GVHD occurs after this initial 100 days. In addition to chronology, different clinical symptoms are also manifest in acute GVHD versus chronic GVHD. Acute GVHD is generally characterized by damage to host liver, skin, mucosa and intestinal epithelium in the host subject, although some forms of idiopathic pneumonia have also been reported. Chronic GVHD is, on the other hand, associated with damage to connective tissue as well as the organs and tissues damaged during acute GVHD in the host subject. In general, the methods of the present invention relate to therapies for either addressing GVHD that is already present in a host subject or preventing GVHD from arising in a host subject. In one embodiment, the present invention relates to methods of treating or preventing acute GVHD. In one specific embodiment, the methods relate to treating acute GVHD where the GVHD is damaging host intestinal epithelium. In other specific embodiments, the methods relate to treating acute GVHD where the GVHD is damaging at least one tissue selected from the group consisting of the host liver, the host skin, the host lung and the host mucosa. Of course, the methods may be used to treat acute GVHD where the GVHD is damaging more than tissue.

In another embodiment, the methods of the present invention relate to methods of treating or preventing chronic GVHD. In one specific embodiment, the methods relate to treating chronic GVHD where the GVHD is damaging host intestinal epithelium. In other specific embodiments, the methods relate to treating chronic GVHD where the GVHD is damaging at least one tissue selected from the group consisting of the host liver, the host skin, the host lung, the host connective tissue and the host mucosa. Of course, the methods may be used to treat acute GVHD where the GVHD is damaging more than tissue.

The term “therapy” is used to indicate a procedure or medicinal regimen designed to ameliorate one or more causes, symptoms, or untoward effects of GVHD in a host subject. The therapy can, but need not, cure the subject, i.e., remove the cause(s), or remove entirely the symptom(s) and/or untoward effect(s) of GVHD in the host subject.

“Prevention” on the other hand is intended to mean a procedure or therapy that is administered or performed prior to the onset of any symptom or diagnosis of GVHD. Thus, therapeutics may be administered in advance of any visible or detectable symptom or indication of GVHD. The prophylactic administration of the substance serves to attenuate subsequently arising symptoms or possibly prevent symptoms from arising altogether in a host subject that may or may not be at risk.

The phrase “treating the host” includes, but is not limited to, treating the host tissue that is damaged or suspected of being susceptible to damage during GVHD, such as intestinal epithelium, liver, mucosa, the lungs, skin and connective tissues thereof. Thus, the methods of the present invention may comprise administering a therapeutic regimen to a subject suffering from GVHD or to a subject that is susceptible to GVHD.

As used herein, the term “administer” and “administering” are used to mean introducing at least one compound into a subject. When administration is for the purpose of treatment, the substance is provided at, or after the onset of, a diagnosis of GVHD. The treatment serves to attenuate any symptom, or prevent additional symptoms from arising. The route of administration of the composition includes, but is not limited to, topical, transdermal, intranasal, vaginal, rectal, oral, subcutaneous intravenous, intraarterial, intramuscular, intraosseous, intraperitoneal, epidural and intrathecal.

Furthermore, the methods of treating or preventing GVHD also relate to coadministering one or more substances in addition to compounds that reduce levels of active CD103. The term “co-administer” indicates that each of at least two compounds is administered during a time frame wherein the respective periods of biological activity or effects overlap. Thus the term includes sequential as well as coextensive administration of the compounds of the present invention. And similar to administering compounds, coadministration of more than one substance can be for therapeutic and/or prophylactic purposes. If more than one substance is coadministered, the routes of administration of the two or more substances need not be the same. In addition, the term co-administer also includes administering a therapeutic regimen to the host and separately treating the graft. The scope of the invention is not limited by the identity of the substance which may be coadministered. For example, antibodies of the present invention may be coadministered with another pharmaceutically active substance, such as but not limited to, methotrexate and cyclosporine. Additional agents that may be co-administered include but are not limited to, antibodies directed to various targets, tacrolimus, sirolimus, interferons, opioids, TNF-α (tumor necrosis factor-α) binding proteins, Mycophenolate mofetil and other inhibitors of inosine monophosphate dehydrogenase (IMPDH), glucocorticoids, azathioprine and other cytostatic agents such as, but not limited to, antimetabolites and alkylating agents, to name a few. In one embodiment, the graft or donor may be pretreated by administration of immunosuppressive drugs such as cyclosporine (alone or in combination with steroids) and methotrexate prior to transplantation. For prevention, immunosuppressive therapy typically consists of combined regimens of methotrexate (MTX), cyclosporin (CsA), tacrolimus (FK 506), and/or a corticosteriod. Intravenous gamma-globulin preparations administered prophylactically have also been shown to be beneficial for the prevention of GVHD. In addition, Pentoxyfylline, a xanthine derivative capable of down-regulating TNF-α production, may be administered with cyclosporin plus either methotrexate or methylprednisolone to further decrease incidence of GVHD. Chronic GVHD may be treated with steroids such as prednisone, ozothioprine and cyclosporine. Also, antithymocyte globulin (ATG) and/or Ursodiol may be used. Thalidomide with immunosuppressive properties has shown promising results in the treatment of chronic GVHD. Similar to thalidomide, clofazimine may also be coadministered with compounds that reduce active levels of CD103. Antibody targets for co-administered antibodies include, but are not limited to, T cell receptor (TCR) and interleukin-2 (IL-2) and IL-2 receptors. Additionally, a CD(25) monoclonal antibody or anti-CD8 monoclonal antibody may be co-administered for GVHD prophylaxis.

Certain of the methods of the present invention are intended to reduce the activity of CD103 in cell originating from the graft. CD103, formerly known as αE integrin is present on both CD4+ T cells (helper T cells) and CD8+ T cells (cytolytic T cells, or CTLs) and serves as a receptor for E-cadherin. E-cadherin is an epithelial-cell specific ligand involved in cell adhesion. As used herein, “reducing the activity of CD103” is intended to mean reducing or decreasing the expression, activation or binding of CD103 on cells normally expressing the molecule. The reduction in activity may be a qualitative or quantitative reduction, and it may or may not include a complete removal of detectable activity. “Decreasing the expression” includes methods designed to decrease the production or post-translational processing of the protein, as well as methods that cleave all or a portion of the extracellular portion of CD103 such that the molecule is not able to bind to E-cadherin with the same affinity as would be normal without therapeutic intervention. By “decreasing” or “reducing” is meant a reduction in activity as compared to the same clinical situation without therapeutic intervention. Decreasing the activity of CD103 also includes methods designed to selectively destroy cells, selectively inhibit or prevent mitosis in cells that normally express CD103 as well as methods of preventing or inhibiting the retention of cells in tissues where CD103-expressing cells are known to adhere, e.g., intestinal epithelium. In one embodiment, the present invention is directed to reducing the activity of CD103 in specific cell populations within the graft. In another embodiment, the present invention is directed to reducing the activity of CD103 in the intestine of the host subject. In a particular embodiment, the methods are directed to reducing the activity of CD103 in CD8+ cells that originate from the graft. In yet another embodiment, the present invention relates to methods of reducing the ability of host cells to interact with CD103-expressing cells originating in the graft.

Methods of determining a reduction or decrease in integrin, e.g., CD103, activity are well known in the art. Examples of assessing a reduction in CD103 activity include, but are not limited to, flow cytomerty, immunoassays, protein and RNA detection methods such as Western blots and Northern blots. Other means for assessing CD103 activity include, but are not limited to, assessing biological activities of cells expressing CD103 such as chemotactic, cytotoxic, enzymatic assays. Other indirect methods for assessing activities of CD103 include, for example, methods employed by the National Institute for Biological Standards and Control (NIBSC) in the United Kingdom for the quantification of interferon, cytokine and growth-factor activity. Such methods may include assessing levels of effector molecules that are secreted from cells that normally express CD103. For example, depleting or reducing the number of CD103 cells present in the graft, which is included in the definition of “reducing CD103 activity,” may be assessed by measuring certain cytokines such as, but not limited to, interferon gamma (IFN-γ) and tumor necrosis factor alpha (TNF-α), that are secreted by select CD103+ cells. CD103+ cells may also secrete chemokines and metaoproteinases. Animal models of GVHD that are predicative of efficacy in target host organisms may also be used to assess the reduction of CD103 in response to a therapeutic regimen or procedure. Still more ways of assessing CD103 activity include ex vivo assays of patient peripheral blood lymphocyte function and/or phenotype. Such methods are disclosed in Hadley et al., Transplantation 16:1418-1425 (1999), which is incorporated by reference.

In one embodiment, the methods of the present invention relate to administering antibody conjugates to treat the graft to reduce the activity of CD103 in cells originating from the graft. In another embodiment, the antibody conjugates bind to CD103.

The present invention also relates to treating the graft comprising co-administering antagonists to transforming growth factor beta (TGF-β). In particular, the antagonists to TGF-β include, but are not limited to, antibodies or binding agents that bind TGF-β receptors. In a more specific embodiment, the antibodies or binding agents may be directed to TGF-β receptors that are expressed on CD8+ cells. In yet another embodiment, the antibodies or binding agents bind to transforming growth factor-β (TGF-β).

As used herein, the term “antibody” is used to mean immunoglobulin molecules and functional fragments thereof, regardless of the source or method of producing the fragment. As used herein, a “functional fragment” of an immunoglobulin is a portion of the immunoglobulin molecule that specifically binds to a binding target. Thus, the term “antibody” as used herein encompasses whole antibodies, such as antibodies with isotypes that include but are not limited to IgG, IgM, IgA, IgD, IgE and IgY, and even single-chain antibodies found in some animals e.g., camels, as well as fragments that specifically bind to target. Whole antibodies thereof may be monoclonal or polyclonal, and they may be humanized or chimeric. The term “monoclonal antibody” as used herein is not limited to antibodies produced through hybridoma technology. Rather, the term “monoclonal antibody” refers to an antibody that is derived from a single clone, including any eukaryotic, prokaryotic, or phage clone, and not the method by which it is produced. The term “antibody” also encompasses functional fragments of immunoglobulins, including but not limited to Fab fragments, Fab′ fragments, F(ab′)₂ fragments and Fd fragments. “Antibody” also encompasses fragments of immunoglobulins that comprise at least a portion of a V_{L} and/or V_{H} domain, such as single chain antibodies, a single-chain Fv (scFv), disulfide-linked Fvs and the like.

The antibodies used in the present invention may be monospecific, bispecific, trispecific or of even greater multispecificity. In addition the antibodies may be monovalent, bivalent, trivalent or of even greater multivalency. Furthermore, the antibodies of the invention may be from any animal origin including, but not limited to, birds and mammals. In specific embodiments, the antibodies are human, murine, rat, sheep, rabbit, goat, guinea pig, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described in U.S. Pat. No. 5,939,598, which is herein incorporated by reference.

The antibodies used in the present invention may be described or specified in terms of the epitope(s) or portion(s) of a polypeptide to which they recognize or specifically bind. Or the antibodies may be described based upon their ability to bind to specific conformations of the antigen, or specific modification (e.g., cleavage or chemical, natural or otherwise, modification of sequence).

The specificity of the antibodies used in present invention may also be described or specified in terms their binding affinity towards the antigen (epitope) or of by their cross-reactivity. Specific examples of binding affinities encompassed in the present invention include but are not limited to those with a dissociation constant (Kd) less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴M, 10⁻¹⁴ M, 5×10⁻¹⁵ M, or 10⁻¹⁵ M.

The antibodies used in the invention also include derivatives that are modified, for example, by covalent attachment of any type of molecule to the antibody such that covalent attachment does not prevent the antibody from generating an anti-idiotypic response. Examples of modifications to antibodies include but are not limited to, glycosylation, acetylation, pegylation, phosphorylation, amidation, derivatization by known protecting/blocking groups, proteolytic cleavage, linkage to a cellular ligand or other composition, such as a signaling moiety, a label etc. In addition, the antibodies may be linked or attached to solid substrates, such as, but not limited to, beads, particles, glass surfaces, plastic surfaces, ceramic surfaces, metal surfaces. Any of numerous chemical modifications may be carried out by known techniques, including, but not limited to, specific chemical cleavage, acetylation, biotinylation, farnesylation, formylation, inhibition of glycosylation by tunicamycin and the like. Additionally, the derivative may contain one or more non-classical or synthetic amino acids.

The antibodies used in the present invention may be generated by any suitable method known in the art. Polyclonal antibodies can be produced by various procedures well known in the art. For example, the antigen or fragment thereof can be administered to various host animals including, but not limited to, rabbits, goats, chickens, mice, rats, to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (both of which are incorporated by reference).

Methods for producing and screening for specific antibodies using hybridoma technology are routine and well known in the art such as, but not limited to, immunizing a mouse, hamster, or rat. Additionally, newer methods to produce rabbit and other mammalian monoclonal antibodies may be available to produce and screen for antibodies. In short, methods of producing and screening antibodies, and the animals used therein, should not limit the scope of the invention. Once an immune response is detected, the mouse spleen is harvested and splenocytes isolated. The splenocytes are then fused by well known techniques to any suitable myeloma cells, for example cells from cell line SP2/0 available from the ATCC. Hybridomas are selected and cloned by limited dilution. The hybridoma clones can then be assayed by methods known in the art for cells that secrete antibodies capable of binding an antigen of the present invention. Ascites fluid, which generally contains high levels of antibodies, can be generated by immunizing mice with positive hybridoma clones. In addition, antibodies can be produced using a variety of alternate methods, including but not limited to bioreactors and standard tissue culture methods, to name a few.

The antibodies used the present invention can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library. Phage expressing an antigen binding domain that binds the antigen of interest can be selected or identified with the antigen of interest, such as using a labeled antigen or antigen bound or captured to a solid surface or bead. The phage used in these methods are typically filamentous phage including, but not limited to, fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108, all of which are incorporated by reference.

Antibody fragments which recognize specific epitopes may be generated by known techniques. For example, Fab and F(ab′)₂ fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain.

Other methods, such as recombinant techniques, may be used to produce Fab, Fab′ and F(ab′)₂ fragments and are disclosed in PCT publication WO 92/22324; Mullinax et al., BioTechniques 12(6):864-869 (1992); and Sawai et al., AJRI 34:26-34 (1995); and Better et al., Science 240:1041-1043 (1988), which are herein incorporated by reference. After phage selection, for example, the antibody coding regions from the phage can be isolated and used to generate whole antibodies, including human antibodies, or any other desired antigen binding fragment, and expressed in any desired host, including mammalian cells, insect cells, plant cells, yeast, and bacteria.

Examples of techniques which can be used to produce other types of fragments, such as scFvs and include those described in U.S. Pat. Nos. 4,946,778 and 5,258,498; Huston et al., Methods in Enzymology 203:46-88 (1991); Shu et al., Proc. Nat'l Acad. Sci. (USA) 90:7995-7999 (1993); and Skerra et al., Science 240:1038-1040 (1988), all of which are incorporated by reference. For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816,397, all of which are herein incorporated by reference. Humanized antibodies are antibody molecules from non-human species antibody that bind the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See U.S. Pat. No. 5,585,089; Riechmann et al., Nature 332:323 (1988), both of which are herein incorporated by reference. Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., Proc. Nat'l. Acad. Sci. 91:969-913 (1994)), and chain shuffling (U.S. Pat. No. 5,565,332), all of which are hereby incorporated by reference.

Completely human antibodies may be particularly desirable for therapeutic treatment or diagnosis of human patients. Human antibodies can be made by a variety of methods known in the art including phage display methods described above using antibody libraries derived from human immunoglobulin sequences. See also. U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated by reference.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995), which is hereby incorporated by reference. For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference.

Still another approach for generating human antibodies utilizes a technique referred to as guided selection. In guided selection, a selected non-human monoclonal antibody, e.g., a mouse antibody, is used to guide the selection of a completely human antibody recognizing the same epitope. (Jespers et al., Biotechnology 12:899-903 (1988), herein incorporated by reference).

Various methods can be used to assess the functional activity of antibodies and fragments. For example, in one embodiment where one is assaying for the ability to bind or compete with a polypeptide for binding to, for example, CD103, various immunoassays known in the art can be used, including but not limited to, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

The antibodies of the present invention must bind an antigen comprising at least a portion of the antigen of interest. The terms “antigen” and “biomarker” are also used interchangeably, as they relate the antibodies and the methods of use described herein. Thus the term “antigen” does not necessarily mean that the biomarkers of the present invention elicit an immune response. In one specific embodiment, the antigen is a polypeptide of comprising an amino acid sequence at least 80%, 85%, 90%, 95%, 97%, 98%, 99% or 100% identical to the amino acid sequence of SEQ ID NO:1, below.

| (SEQ ID NO: 1) | |
|---|---|
| 1 | mwlfhtllci aslallaafn vdvarpwltp kggapfvlss llhqdpstnq twllvtsprt |
| 61 | krtpgplhrc slvqdeilch pvehvpipkg rhrgvtvvrs hhgvliciqv lvrrphslss |
| 121 | eltgtcsllg pdlrpqaqan ffdlenlldp darvdtgdcy snkegggedd vntarqrral |
| 181 | ekeeeedkee eedeeeeeag teiaiildgs gsidppdfqr akdfisnmmr nfyekcfecn |
| 241 | falvqyggvi qtefdlrdsq dvmaslarvq nitqvgsvtk tasamqhvld siftsshgsr |
| 301 | rkaskvmvvl tdggifedpl nlttvinspk mqgverfaig vgeefksart arelnliasd |
| 361 | pdethafkvt nymaldglls klryniisme gtvgdalhyq laqigfsaqi lderqvllga |
| 421 | vgafdwsgga llydtrsrrg rflnqtaaaa adaeaaqysy lgyavavlhk tcslsyvaga |
| 481 | pqykhhgavf elqkegreas flpvlegeqm gsyfgselcp vdidmdgstd fllvaapfyh |
| 541 | vhgeegrvyv yrlseqdgsf slarilsghp gftnarfgfa maamgdlsqd kltdvaigap |
| 601 | legfgaddga sfgsvyiyng hwdglsasps qrirastvap glqyfgmsma ggfdisgdgl |
| 661 | aditvgtlgq avvfrsrpvv rlkvsmaftp salpigfngv vnvrlcfeis svttasesgl |
| 721 | reallnftld vdvgkqrrrl qcsdvrsclg clrewssgsq lcedlllmpt egelceedcf |
| 781 | snasvkvsyq lqtpegqtdh pqpildryte pfaifqlpye kacknklfcv aelqlattvs |
| 841 | qqelvvgltk eltlninltn sgedsymtsm alnyprnlql krmqkppspn iqcddpqpva |
| 901 | svlimncrig hpvlkrssah vsvvwqleen afpnrtadit vtvtnsnerr slanethtlq |
| 961 | frhgfvavls kpsimyvntg qglshhkefl fhvhgenlfg aeyqlqicvp tklrglqvaa |
| 1021 | vkkltrtqas tvctwsqera cayssvqhve ewhsvscvia sdkenvtvaa eiswdhseel |
| 1081 | lkdvtelqil geisfnksly eglnaenhrt kitvvflkde kyhslpiiik gsvggllvli |
| 1141 | vilvilfkcg ffkrkyqqln lesirkaqlk senlleeen |

In another specific embodiment, the antigen is a polypeptide of comprising an amino acid sequence at least 80%, 85%, 90%, 95%, 97%, 98%, 99% or 100% identical to residues corresponding to residues 2-1179 of the amino acid sequence of SEQ ID NO:1. In another specific embodiment, the antigen is a polypeptide of comprising an amino acid sequence at least 80%, 85%, 90%, 95%, 97%, 98%, 99% or 100% identical to residues corresponding to residues 19-1179 of the amino acid sequence of SEQ ID NO:1. In another specific embodiment, the antigen is a polypeptide of comprising an amino acid sequence at least 80%, 85%, 90%, 95%, 97%, 98%, 99% or 100% identical to residues corresponding to residues 19-176 of the amino acid sequence of SEQ ID NO:1. In another specific embodiment, the antigen is a polypeptide of comprising an amino acid sequence at least 80%, 85%, 90%, 95%, 97%, 98%, 99% or 100% identical to residues corresponding to residues 177-1179 of the amino acid sequence of SEQ ID NO:1. In another specific embodiment, the antigen is a polypeptide of comprising an amino acid sequence at least 80%, 85%, 90%, 95%, 97%, 98%, 99% or 100% identical to residues corresponding to residues 144-198 of the amino acid sequence of SEQ ID NO:1. In another specific embodiment, the antigen is a polypeptide of comprising an amino acid sequence at least 80%, 85%, 90%, 95%, 97%, 98%, 99% or 100% identical to residues corresponding to residues 199-390 of the amino acid sequence of SEQ ID NO:1. In another specific embodiment, the antigen is a polypeptide of comprising an amino acid sequence at least 80%, 85%, 90%, 95%, 97%, 98%, 99% or 100% identical to residues corresponding to residues 201-378 of the amino acid sequence of SEQ ID NO:1. In another specific embodiment, the antigen is a polypeptide of comprising an amino acid sequence at least 80%, 85%, 90%, 95%, 97%, 98%, 99% or 100% identical to residues corresponding to residues 510-565 of the amino acid sequence of SEQ ID NO:1. In another specific embodiment, the antigen is a polypeptide of comprising an amino acid sequence at least 80%, 85%, 90%, 95%, 97%, 98%, 99% or 100% identical to residues corresponding to residues 574-634 of the amino acid sequence of SEQ ID NO:1.

As used herein, “identity” as it relates to amino acid sequence or polynucleotide sequences is a measure of the identity of nucleotide sequences or amino acid sequences compared to a reference nucleotide or amino acid sequence, usually a wild-type sequence. In general, the sequences are aligned so that the highest order match is obtained. “Identity” per se has an art-recognized meaning and can be calculated using published techniques. (See, e.g., Computational Molecular Biology, Lesk, A. M., ed., Oxford University Press, New York (1988); Biocomputing: Informatics And Genome Projects, Smith, D. W., ed., Academic Press, New York (1993); Computer Analysis of Sequence Data, Part I, Griffin, A. M., and Griffin, H. G., eds., Humana Press, New Jersey (1994); von Heinje, G., Sequence Analysis In Molecular Biology, Academic Press (1987); and Sequence Analysis Primer, Gribskov, M. and Devereux, J., eds., M Stockton Press, New York (1991)). While several methods exist to measure identity between two polynucleotide or polypeptide sequences, the term “identity” is well known in the art (Carillo, H. & Lipton, D., Siam J Applied Math 48:1073 (1988)). Methods commonly employed to determine identity or similarity between two sequences include, but are not limited to, those disclosed in Guide to Huge Computers, Martin J. Bishop, ed., Academic Press, San Diego (1994) and Carillo, H. & Lipton, D., Siam J Applied Math 48:1073 (1988). Computer programs may also contain methods and algorithms that calculate identity and similarity. Examples of computer program methods to determine identity and similarity between two sequences include, but are not limited to, GCS program package (Devereux, J., et al., Nucleic Acids Research 12(i):387 (1984)), BLASTP, BLASTN, FASTA (Atschul, S. F., et al., J Molec Biol 215:403 (1990)).

A polypeptide having an amino acid sequence at least, for example, about 95% “identical” to a reference nucleotide sequence encoding a peptide of interest, for example CD103 or an antibody to CD103, is understood to mean that the amino acid sequence of the peptide is identical to the reference sequence except that the amino acid sequence may include up to about five mutations per each 100 amino acids of the reference peptide sequence encoding the peptide being used as the reference sequence. In other words, to obtain a polypeptide having an amino acid sequence at least about 95% identical to a reference amino acid sequence, up to about 5% of the amino acids in the reference sequence may be deleted or substituted with another amino acid, or a number of amino acids up to about 5% of the total amino acids in the reference sequence may be inserted into the reference sequence. These mutations of the reference sequence may occur at the N- or C-terminal positions of the reference amino acid sequence or anywhere between those terminal positions, interspersed either individually among amino acids in the reference sequence or in one or more contiguous groups within the reference sequence.

As used herein, the terms “correspond(s) to” and “corresponding to,” as they relate to sequence alignment, are intended to mean enumerated positions within a reference protein, e.g., wild-type human CD103, and those positions in that align with the positions on the reference protein in a sequence alignment. Thus, when the amino acid sequence of an antigen or biomarker is aligned with the amino acid sequence of a reference CD103, e.g., SEQ ID NO:1, the amino acids in the subject antigen sequence that “correspond to” certain enumerated positions of the reference CD103 sequence are those that align with these positions of the reference CD103 sequence, but are not necessarily in these exact numerical positions of the reference CD103 sequence. Methods for aligning sequences for determining corresponding amino acids between sequences are described below.

The present invention also relates to isolated nucleic acids encoding the antibodies or binding agents of the present invention. As used herein, “isolated” as it relates to a polypeptide or nucleic acid molecule, is used to mean a polypeptide or nucleic acid molecule that has been removed from its native environment. For example, polypeptides that have been removed or purified from cells are considered isolated. In addition, recombinantly produced polypeptides molecules contained in recombinant host cells are considered isolated for the purposes of the present invention. Similarly, recombinant DNA molecules contained in a vector are considered isolated for the purposes of the present invention. Further examples of isolated DNA molecules include, but are not limited to, recombinant DNA molecules maintained in recombinant host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Nucleic acid molecules of the present invention may be in the form of RNA, such as but not limited to mRNA, or in the form of DNA, including, for instance, cDNA and genomic DNA obtained by cloning or produced synthetically. The DNA may be, but is not limited to, double-stranded or single-stranded. Single-stranded DNA or RNA may be the coding strand, also known as the sense strand, or it may be the non-coding strand, also referred to as the anti-sense strand.

Using the information provided herein, a nucleic acid molecule of the present invention encoding an antibody specific for CD103 biomarker may be obtained using standard cloning and screening procedures, such as those for cloning cDNAs using mRNA as starting material.

Nucleotide sequences can be determined using an automated DNA sequencer (such as the Model 373 from Applied Biosystems, Inc.). Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

By a polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence encoding an antibody to, for example, CD103. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As used herein, “identity” is a measure of the identity of nucleotide sequences or amino acid sequences compared to a reference nucleotide or amino acid sequence. In general, the sequences are aligned so that the highest order match is obtained. “Identity” per se has an art-recognized meaning and can be calculated using published techniques. (See, e.g., Computational Molecular Biology, Lesk, A. M., ed., Oxford University Press, New York (1988); Biocomputing: Informatics And Genome Projects, Smith, D. W., ed., Academic Press, New York (1993); Computer Analysis of Sequence Data, Part I, Griffin, A. M., and Griffin, H. G., eds., Humana Press, New Jersey (1994); von Heinje, G., Sequence Analysis In Molecular Biology, Academic Press (1987); and Sequence Analysis Primer, Gribskov, M. and Devereux, J., eds., M Stockton Press, New York (1991)). The term “identity” is well known to skilled artisans, and several methods exist to measure identity between two polynucleotide or polypeptide sequences (Carillo, H. & Lipton, D., Siam J Applied Math 48:1073 (1988)). Methods commonly employed to determine identity or similarity between two sequences include, but are not limited to, those disclosed in Guide to Huge Computers, Martin J. Bishop, ed., Academic Press, San Diego (1994) and Carillo, H. & Lipton, D., Siam Applied Math 48:1073 (1988). Computer programs may also contain methods and algorithms that calculate identity and similarity. Examples of computer program methods to determine identity and similarity between two sequences include, but are not limited to, GCG program package (Devereux, J., et al., Nucleic Acids Research 12(i):387 (1984)), BLASTP, ExPASy, BLASTN, FASTA (Atschul, S. F., et al., J Molec Biol 215:403 (1990)) and FASTDB. Examples of methods to determine identity and similarity are discussed in Michaels, G. and Garian, R., Current Protocols in Protein Science, Vol 1, John Wiley & Sons, Inc. (2000), which is incorporated by reference.

In one embodiment, the method for determining the overall match between a query sequence (a sequence of the present invention) and a subject sequence, also referred to as a global sequence alignment, comprises the use of the FASTDB computer program based on the algorithm of Brutlag et al. (Comp. App. Biosci. 6:237-245 (1990)). In one example, the sequence alignment the query and subject sequences are both DNA sequences. An RNA sequence can be compared by converting U's to T's. The result of said global sequence alignment is in percent identity. Parameters that may be used in a FASTDB alignment of DNA sequences to calculate percent identity include, but are not limited to: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter.

If the subject sequence is shorter than the query sequence because of 5′ or 3′ deletions, not because of internal deletions, a manual correction can be made because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Nucleotide matching/alignment is determined by results of the FASTDB sequence alignment. The alignment percentage is then subtracted from the percent identity, calculated by the above FASTDB program using the specified parameters, to arrive at a final percent identity score. This corrected score can be used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. In this example, the deletions occur at the 5′ end of the subject sequence and therefore, the FASTDB alignment does not show a match/alignment of the first 10 bases at the 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected.

Of course, due to the degeneracy of the genetic code, one of ordinary skill in the art will immediately recognize that a large number of the nucleic acid molecules having a sequence at least 80%, 85%, 90%, 92%, 95%, 96%, 97%, 98% or 99% identical to the reference nucleic acid can encode a polypeptide that may bind an antigen, e.g., CD103. In fact, since degenerate variants of these nucleotide sequences all encode the same polypeptide, this will be clear to the skilled artisan even without performing the above described comparison assay.

In one embodiment of the present invention, the algorithm used to determine identity between two or more polypeptides is BLASTP. In another embodiment of the present invention, the algorithm used to determine identity between two or more polypeptides is FASTDB. In this particular method of using the FASTDB algorithm for a sequence alignment, the query and subject sequences are amino sequences. The result of sequence alignment is in percent identity. Parameters that may be used in a FASTDB alignment of amino acid sequences to calculate percent identity include, but are not limited to: Matrix=PAM, k-tuple=2, Mismatch Penalty=1, Joining Penalty=20, Randomization Group Length=0, Cutoff Score=1, Gap Penalty=5, Gap Size Penalty 0.05, Window Size=500 or the length of the subject amino sequence, whichever is shorter.

As used herein, a lethal compound is a compound that induces cell death in any fashion, including but not limited to necrosis, apoptosis and cytotoxicity. Examples of lethal compounds include but are not limited to, apoptosis inducing proteins, but not limited to p53, Rb and BRCA-1, toxins such as diphtheria toxin (DTA), shigella neurotoxin, botulism toxin, tetanus toxin, cholera toxin, saporin, ricin, CSE-V2, and other variants of scorpion protein toxins to name a few. In one embodiment, however, the lethal compound is saporin. Other lethal substances are well known and can be found in publications such as, but not limited to, The Merck Index.

The present invention should not be limited by the identity of the lethal compound, provided that the lethal compound is capable of being lethal to the CD103+ cell once it is delivered to the cell. Indeed, other examples of lethal compounds include, but are not limited to, radionuclides. Any type of radioactivity, e.g., alpha and beta particles, gamma rays, X-rays and the like, can be used, provided the radioactivity is lethal to the cell to which it is delivered. In one particular embodiment, the lethal compound emits radioactive alpha particles.

Any conventional method of radiolabeling, which is suitable for labeling proteins for in vivo use, generally is suitable for labeling the antibody conjugates. Labeling can be achieved directly with, for example, ¹³¹I, ¹²⁴I or ¹²³I. Labeling with either ¹³¹I, ¹²⁴I or ¹²³I, is readily effected using an oxidative procedure wherein a mixture of radioactive potassium or sodium iodide and the antibody is treated with chloramine-T, as reported by Greenwood et al., Biochem. J., 89: 114 (1963) and modified by McConahey et al., Int. Arch. Allergy Appl. Immunol., 29: 185 (1969). The oxidative procedure results in direct substitution of iodine atoms for hydrogen atoms on the antibody molecule. Alternatively, either Iodogen-based methods, as described by Fraker et al., Biochem Biophys Res. Commun. 80:849-857, 1978, or lactoperoxidase iodination may be used.

Instead of ¹³¹I, ¹²⁴I or ¹²³I, the conjugate can be labeled by metallation with, for example, ^{99m}Tc or Cu ions or the like, by conventional techniques, or by attaching a chelator for a radiometal or paramagnetic ion. Such chelators and their modes of attachment to antibodies are well known to the skilled artisan.

Examples of radionuclides include but are not limited to, iodine-123, iodine-124, iodine-131, indium-111, gallium-67, gallium-68, ruthenium-97, technetium-94, technetium-99m, copper-64, copper-67, cobalt-57, cobalt-58, chromium-51, iron-59, yttrium-86, selenium-75, thallium-201, ytterbium-169, bismuth-212, astatine-211, yttrium-90, rhenium-186, rhenium-188, copper-67 and iodine-131. Additional examples of radionuclides include, but are not limited to, electron capture or Auger conversion electron-emitting radionuclides such as iodine-125, indium-111 and gallium 67.

The invention may also be used to screen antibodies, antibody conjugates or other compounds that have been developed as potential therapeutics, such as, but not limited to, humanized antibodies and antagonists to CD103 and/or TGF-β. Vaccination studies may be undertaken that have the intent of generating antibodies in the subject that bind and antagonize the effects of the antigen of the present invention. The antibodies of the present invention may be used to compare the affinity or other characteristics of generated antibodies or other molecules, such as candidate drugs or compositions that may bind a target molecule, such as, but not limited to, TGF-β, TGF-β receptor, CD103 and E-cadherin. Specifically, the invention provides methods of assessing the affinity of binding agents, e.g., antibodies, comprising competition assays between the antibodies of the present invention and a different binding agent.

In one embodiment, antibody or binding agent binding is detected by detecting a label on the antibody or primary binding agent. In another embodiment, the primary antibody or biding agent is detected by detecting binding of a secondary antibody or reagent to the primary antibody or primary binding agent. In a further embodiment, the secondary antibody is labeled. Many means are known in the art for detecting binding in an immunoassay and are within the scope of the present invention. In particular embodiments of the present invention, an antibody would be bound to a solid support (such as glass, polystyrene, polypropylene, polyethylene, dextran, nylon, amylases, natural and modified celluloses, polyacrylamides, agaroses, and magnetite). The nature of the carrier can be either soluble to some extent or insoluble for the purposes of the present invention. The support material may have virtually any possible structural configuration so long as the coupled molecule is capable of binding to the antigen. Thus, the support configuration may be spherical, as in a bead, or cylindrical, as in the inside surface of a test tube, or the external surface of a rod. Alternatively, the surface may be flat such as a sheet, test strip, etc. Those skilled in the art will note many other suitable carriers for binding antibody, or will be able to ascertain the same by use of routine experimentation.

For example, ELISA assays utilize a capture molecule that initially binds to the antigen is or can be bound to the wells of culture plate. In this embodiment, the culture plate is acting as the carrier for the binding agent, e.g., the antibody. Subsequently, a labeled detection molecule (which may recognize the capture molecule or the biomarker) may be added to the test environment. One of skill in the art would be knowledgeable as to the parameters that can be modified to increase the signal detected as well as other variations of ELISAs known in the art. As used herein the term “capture molecule” is used mean a binding agent that immobilizes the antigen by its binding to the antigen. Further, an antigen is “immobilized” if the antigen or antigen-capture molecule complex is separated or is capable of being separated from the remainder of the sample. When the capture molecule is coated to a well or other surface, a detection molecule may be added following the addition of the antigen of interest to the wells. As used herein, a detection molecule is used to mean a molecule, such as an antibody or receptor, comprising a label. In a specific embodiment, the methods of the present invention comprise the use of a capturing antibody and a detection antibody to detect the antigen. In a more specific embodiment, the capture antibody and the detection antibody are the same antibodies with the same binding specificities. In another specific embodiment, the capture antibody and the detection antibody are different antibodies.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is may be the carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, which is incorporated by reference. Such compositions will contain a therapeutically effective amount of the compound, together with a suitable amount of carrier so as to provide the form for proper administration to the patient. The formulation should suit the mode of administration. The parental preparation can be enclosed in ampoules, disposable syringes or multiple dose vials made of glass or plastic.

The composition may be formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment, inhibition and prevention of GVHD may be determined by standard clinical techniques. In addition, in vitro assays of the present invention may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

As a general proposition, the total pharmaceutically effective amount of composition administered parenterally per dose will be in the range of about 1 μg/kg/day to 10 mg/kg/day of patient body weight, although, as noted above, this will be subject to therapeutic discretion. In particular, this dose is at least 0.01 mg/kg/day, and more particularly, for humans, between about 0.01 and 1 mg/kg/day for the hormone. If given continuously, the composition may typically be administered at a dose rate of about 1 μg/kg/hour to about 50 μg/kg/hour, either by 1-4 injections per day or by continuous subcutaneous infusions, for example, using a mini-pump. An intravenous bag solution may also be employed.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration, e.g., into the brain, of the antibodies by modifications such as, for example, lipidation.

The invention also provides a pharmaceutical pack or kit comprising one or more containers filled with one or more of the ingredients of the pharmaceutical compositions of the invention, and, optionally instructions or product insert.

The following examples are meant to illustrate certain embodiments of the present invention and should not be taken as any limit to the scope of the claimed invention.

### EXAMPLES

### Methods and Materials for Examples

Animals—2C T cell receptor transgenic (TCR-tg) mice (H-2^{b}) which express a T cell receptor with specificity towards an 8-mer peptide (p2Ca) Sha, W. C., et al., Nature 335:271-274 (1988); Udaka, K., et al., Proc Natl Acad Sci USA 90:11272-11276 (1993)) were originally obtained from Dr. Ted Hansen (Washington University School of Medicine). The 2C transgenic mice were maintained by backcrossing heterozygous 2C males to C57BL/6 females and screening the offspring for expression of the clonotypic 2C TCR by FACS of peripheral blood using the 1B2 monoclonal antibody (mAb). Mice expressing a dominant negative type II TGF-β receptor (DNR) (Lucas, P. J., et al., J Exp Med 191:1187-1196 (2000)), as well as 2C mice that were bred onto the DNR background (2C-DNR) (NIH, Bethesda, Md., USA) were used in some experiments. C57BL/6 (B6) (H-2^{b}) and BALB/c (H-2^{d}) mice were purchased from Jackson Laboratory (Bar Harbor, Me., USA). 2C CD103^{-/-} mice were generated by breeding the 2C (H-2^{b}) mice and CD103^{-/-} C57BL/6 (H-2^{b}) mice (Schon, M. P., et al., J Immunol 162:6641-6649 (1999)), followed by backcrossing the 2C CD103^{+/-}F1 generation to the CD103^{-/-} parental strain to generate 2C CD103^{-/-} mice. CD103^{-/-} mice were identified by surface staining for the TCR transgene and polymerase chain reaction (PCR) for the homozygous CD103^{-/-} mutation. Wild type congenic 2C Thy1.1⁺ mice were obtained by interbreeding 2C Thy1.2⁺/Thy1.2⁺ and C57BL/6 Thy1.1⁺/Thy1.1⁺ animals to generate a 2C Thy1.1⁺/Thy1.2⁺ F1 generation. All mice were maintained under specific pathogen-free conditions in the animal facility at the University of Maryland, Baltimore. All animal studies were approved by the Institutional Review Board.

Production of Antibodies Specific for CD103—1B2 mAb (anti-clonotypic 2C TCR; mouse IgGl) (Kranz, D. M., et al., Proc Natl Acad Sci USA 81:573-577 (1984)) was obtained from hybridoma culture supernatant. Biotinylated rat anti-mouse IgGl was purchased from Caltag (Burlingame, Calif., USA). Directly conjugated anti-mouse antibodies used included CD8-FITC, CD8-PerCP, Thy1.1-PerCP, Thy1.2-PE, CD44-FITC, CD44-PE, CD62L-PE, M290 (anti-CD103)-PE, CD11a-PE, CD25-PE, IFN-γ-PE, TNF-α-PE and the species- and isotype-matched controls, all of which were purchased from BD PharMingen (San Diego, Calif. USA).

Adoptive Transfer and Bone Marrow Transplantation—Lethally irradiated (8 Gy, ¹³⁷Cs source) BALB/c (H-2^{d}) mice were reconstituted within 4-6 hours by a single 0.5 ml intravenous inoculum containing 10×10⁶B6 bone marrow cells (BMC) and 10×10⁶α6 spleen cells (SC) along with either 1×10⁶ 2C SC or 1×10⁶ 2C-DNR cells. In some experiments, equal numbers (0.5×10⁶) of wild type Thy1.1⁺ 2C and Thy1.1⁻2C-DNR spleen cells were mixed together and adoptively transferred in combination with B6 BMC and SC into lethally-irradiated BALB/c hosts.

In adoptive transfer experiments intended to elucidate the role of CD103 in lymphocyte migration to the intestine, equal numbers (0.5×10⁶) of wild type Thy1.1⁺2C spleen cells and CD103^{-/-} Thy1.1⁻2C spleen cells were mixed and adoptively transferred into a group of lethally irradiated BALB/c hosts. In some experiments, 1×10⁶ wild type or CD103^{-/-}2C cells were separately transferred into lethally irradiated BALB/c mice in combination with C57BL/6 BM and SC.

To examine the survival and organ pathology in recipients of CD103^{-/-} CD8 cells, two groups of lethally irradiated BALB/c hosts received 10×10⁶B6 BMC and either 10×10⁶ CD8-enriched B6 SC or 10×10⁶ CD8-enriched B6 CD103^{-/-} SC. In these experiments, donor spleen cells were enriched for naive CD8⁺ T cells by treatment with a mAb to CD4 (RL172.4), followed by incubation in 1/10 Low-Tox M rabbit complement (Accurate Chemical and Scientific, Westbury, N.Y., USA). The resulting cell suspensions contained <0.5% CD4⁺ T cells.

To ensure that equivalent numbers of CD8 cells were transferred in different experiments, flow cytometry analysis using anti-CD8 and TCR-αβ was performed on each preparation to assess the degree of enrichment for CD8⁺ T cells. Additional experiments were performed by transferring 10×10⁶B6 BMC alone. Mice were randomly grouped before and after irradiation and were given acidified drinking water to prevent infection. Control irradiated untreated mice were also included in each experiment.

Lymphocyte Isolation—Lymphocytes infiltrating the intestinal epithelium during GVHD were isolated as previously described in Guy-Grand, D., et al., J Exp Med 148:1661-1677 (1978), with modifications. The small intestine was flushed with PBS. After removal of the Peyer's patches and fat, the intestine was divided longitudinally and cut into 2-mm sections. The mucosa was scraped and dissociated by mechanical disruption on a stirring platform for 15 min in RPMI 1640 containing 10% FCS and 1 mM dithioerythritol. Tissue debris and cell aggregates were removed by passage over a glass wool column in RPMI 1640-10% FCS. The lymphocytes were obtained by centrifugation on Lympholyte-M (Cedarlane Laboratories Limited, Hornby, Ontario, CANADA) and the cells were suspended in complete medium.

Lymphocytes were isolated from the kidney as previously described in Wang, D., et al., J Immunol 172:214-221 (2004), incorporated by reference. Briefly, the kidney was minced and the resulting fragments incubated for 30 min in medium containing collagenase (Worthington Biochemical, Freehold, N.J., USA), soybean trypsin inhibitor (Sigma-Aldrich, St. Louis, Mo., USA), and DNase (Roche, Indianapolis, Ind., USA). Lymphocytes were isolated by centrifugation on Lympholyte-M (Cedarlane Laboratories). Cells were isolated from the mesenteric lymph nodes (MLN) and spleen of recipients by mincing with forceps and passage of the resulting cell suspension through nylon mesh of 100-μm pore size. Lung- and liver-infiltrating lymphocytes were harvested as described in Masopust, D., et al., Science 291:2413-2417 (2001), but with modifications. Following perfusion with phosphate-buffered saline (PBS), liver and lung tissue was minced and incubated with stirring at 37° C. for 30 min in medium containing collagenase (Worthington Biochemical), soybean trypsin inhibitor (Sigma-Aldrich), and DNase (Roche). Lymphocytes were isolated by centrifugation on Lympholyte-M (Cedarlane Laboratories). Lymphocyte populations were washed twice in FACS buffer prior to antibody staining and FACS analyses.

Histology—Host organs were harvested at the designated times, fixed in 10% buffered formalin, and embedded in paraffin. Sections (6 μm) were stained with H&E and blindly analyzed.

Flow Cytometry Analysis—For three-color flow cytometry analysis, harvested cells were stained with PerCP-conjugatedmAb to CD8b in combination with FITC- and PE-conjugated mAbs to markers of interest. For four-color flow cytometry analysis, cells were stained with anti-CD8a-FITC and anti-Thy1.1-PerCP in combination with PE- and APC-conjugated antibodies. Species and isotype-matched mAbs of irrelevant specificity were used as controls for non-specific fluorescence. After staining, cells were fixed with 0.5% paraformaldehyde and analyzed using a FACSCalibur (Becton Dickinson, San Jose, Calif., USA).

For intracellular cytokine staining (ICS), harvested cells were activated with PMA and ionomycin, followed by addition of 4 μl/ml of Monensin (Golgi Stop, Pharmingen) for an additional 6 hours at 37° C. Cells were washed and resuspended in staining buffer followed by surface staining Cells were then washed and resuspended in Cytoperm/Cytofix solution (PharMingen) for 20 min on ice and subsequently incubated on ice for 30-60 min with appropriate dilutions of anti-cytokine or isotype control antibodies. For Annexin-V staining, lymphocytes were stained in FACS® buffer at 4° C. with FITC-conjugated mAb to CD8b and APC-conjugated mAb to Thy1.1. Cells were then washed and incubated in Annexin-V binding buffer with PE-Annexin-V at 1:20 dilution and 7AAD (to exclude non-viable cells) for 15 min at room temperature. The cells were washed, resuspended in Annexin-V binding buffer and analyzed within 1 hour.

Lymphocyte populations were gated by forward scatter/side scatter analysis to exclude monocytes and debris. WinMDI 2.8 software developed by Dr. Joseph Trotter (Scripps Institute, San Diego, Calif.) (downloaded from the internet at facs.scripps.edu/software.html) was used for analysis and graphic display of flow cytometry data. The percentage of positive cells for a given marker was based on cutoff points chosen to exclude >99% of the negative control population.

Statistical Analyses—Data are expressed as mean±SEM. To assess statistical significance, Student's t tests were performed using SigmaPlot 2000 software (SPSS Inc., Chicago, Ill., USA). P values of less than 0.05 were considered statistically significant.

### Example 1

### Assessing the Role of CD103 in Graft versus Host Disease

Gut-specific Expression of CD103 by hsCD8eff—To determine if (hsCD8eff) populations express CD103, lethally irradiated BALB/c (L^{d+}) mice received 10×10⁶ BMC and 10×10⁶SC from C57BL/6 (B6) (H2^{b}) donors plus trace numbers of spleen cells from 2C TCR-transgenic (B6) mice. Peripheral CD8+ cells from 2C mice express a TCR with specificity for H-2L^{d}, which is readily detected using a mAb (1B2) directed to the clonotypic 2C TCR. Thus, use of mAbs to CD8 and 1B2 in multi-color FACS analyses allowed the phenotypic attributes of donor CD8 effector populations targeting host L^{d} (host-specific; CD8⁺1B2⁺ cells) to be monitored in vivo during the course of GVHD.

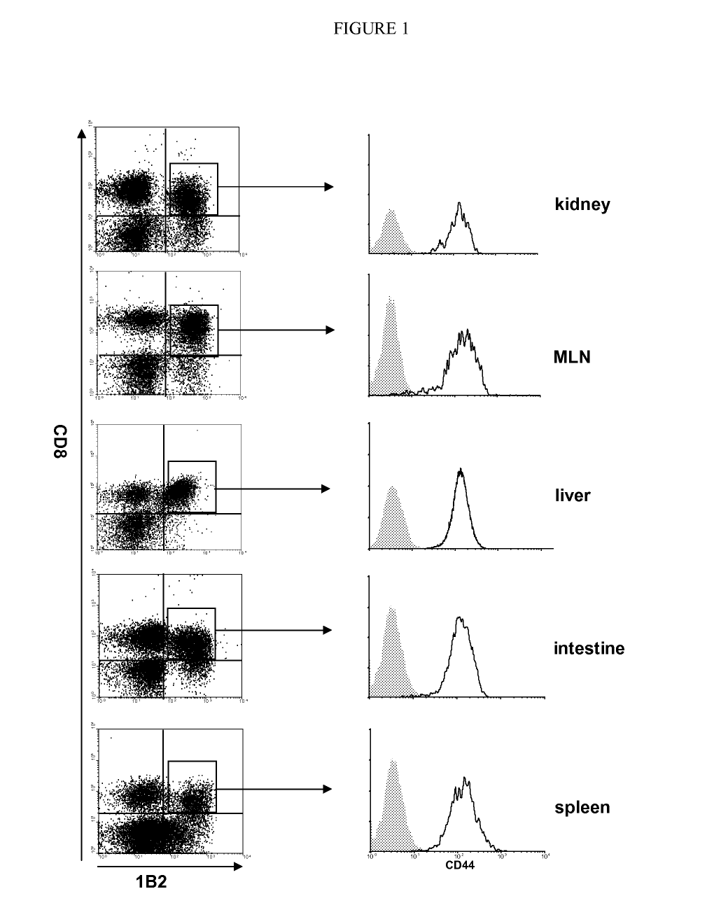

Prior to transfer, the vast majority of host-specific CD8⁺ T cells (1B2⁺CD8⁺) contained within the inoculum were CD44⁻CD11a^{lo}CD62L^{hi} indicating a naive phenotype (data not shown). FIG. 1 shows that, following adoptive transfer into lethally irradiated recipients, 1B2⁺CD8⁺ cells infiltrated host lymphoid (spleen and MLN) and non-lymphoid (kidney, liver and intestine) compartments as early as day 5 post-BMT and were readily detected by day 7. All such cells displayed an effector phenotype)(CD44^{hi}CD11a^{hi}CD62L^{lo}). Migration of donor effector T cells into peripheral compartments correlated with the development of classic manifestations of GVHD, e.g., diarrhea, weight loss, wasting and mortality (MST=7.5±0.3 days; range=6-21 days).

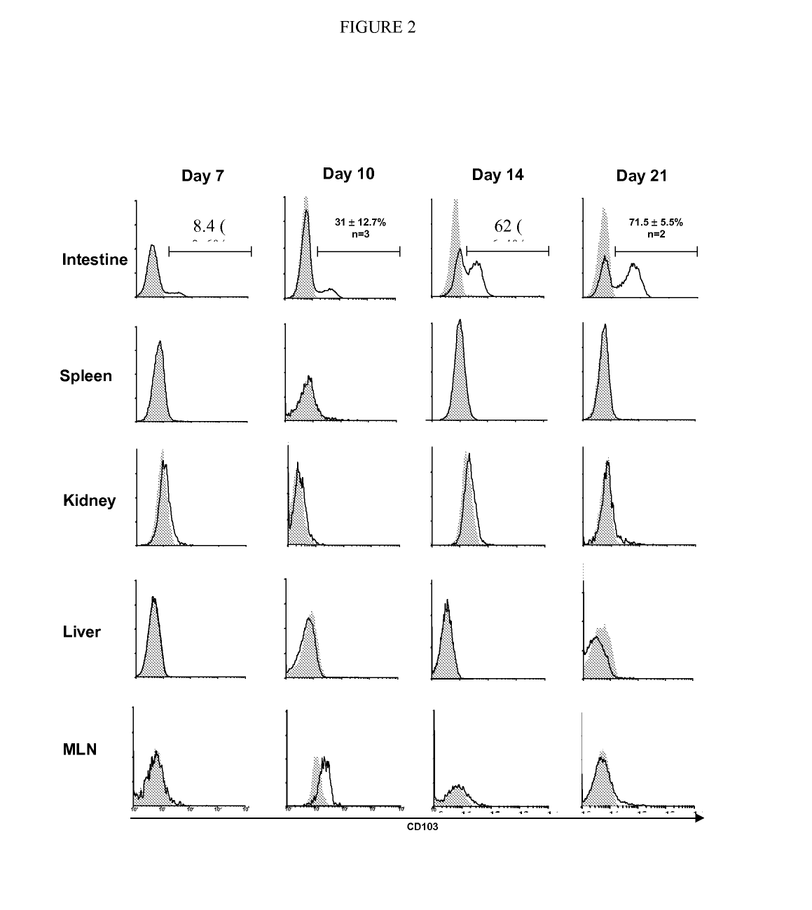

As shown in FIG. 2, a major subset of hsCD8eff that infiltrated the host intestinal epithelium during GVHD expressed significant levels of CD103, the percentage of which progressively increased with time. Thus, at day 7 post-BMT, 8.4±0.6% (n=6) of the 1B2⁺CD8⁺ T cells expressed CD103, and this fraction increased to 31±12.7% (n=3) by day 10, 62±6.4% (n=3) by day 15, and 71.5±5.5% (n=2) by day 21. FIG. 2 also demonstrates that hsCD8eff cells that infiltrated other host compartments, including the spleen, kidney, liver, and MLN, expressed negligible levels of CD103 at all time points examined. The mean percentage ±SE of 1B2⁺CD8⁺T cells in the spleen which expressed significant levels of CD103 was 0.36±0.2, 1.2±0.87, 2.57±1.1, and 0.8±0.2 at days 7, 10, 14, and 21, respectively (all values P<0.05 compared to the intestine).

Non-transgenic CD8 effectors (1B2⁻CD8⁺CD44^{hi} cells) that infiltrated the host intestine also selectively expressed CD103, demonstrating that gut-specific expression of CD103 is not unique to TCR-transgenic CD8 cells (data not shown). Thus, these data revealed that CD103 is selectively expressed by hsCD8eff infiltrating the intestinal epithelium during the course of GVHD.

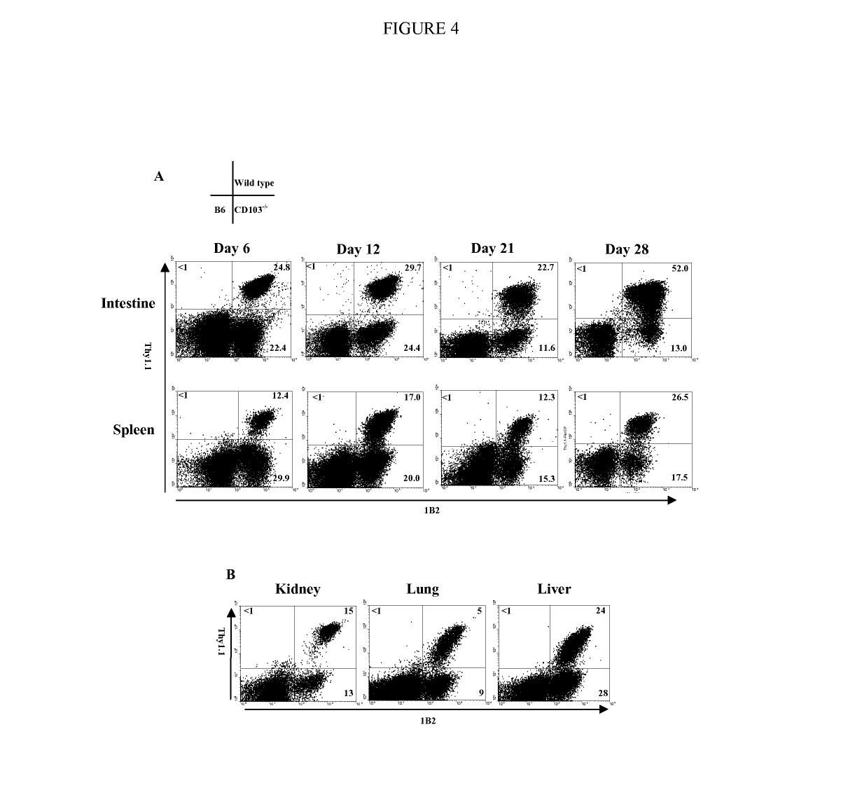

CD103 Expression Promotes Retention of hsCD8eff in the Intestinal Epithelium—To determine if CD103 expression promotes retention of CD103⁺CD8⁺ effectors in the host intestinal epithelium during GVHD, via E-cadherin, 2C mice were crossed onto the CD103^{-/-} background. The resulting CD103^{-/-}2C effectors were compared with wild type counterparts for their capacity to migrate into the host intestinal epithelium. In these experiments, equal numbers (0.5×10⁶) of wild type 2C (Thy1.1⁺1B2⁺) cells and CD103^{-/-}2C (Thy1.1⁻1B2⁺) cells, combined with B6 BM and SC, were transferred into lethally irradiated BALB/c hosts (Thy1.2⁺/Thy1.2⁺). Host organs were harvested at day 6, 12, 21, and 28 post-transfer, and the infiltrating lymphocytes were subjected to four-color FACS® analyses. Prior to transfer CD8⁺ T cells derived from both donor populations displayed a naive phenotype (data not shown), but following transfer both populations rapidly acquired a CD62L^{lo}CD11a^{hi} phenotype characteristic of T effector cells (FIGS. 4A and 4B). Furthermore, wild type and CD103^{-/-}hsCD8eff secreted comparable levels of the effector cytokines TNF-α (FIG. 4C) and IFN-γ (FIG. 4D).

As shown in FIG. 4A (top), CD103^{-/-} hsCD8eff comprised 22% of the total CD8 T cells infiltrating the host intestinal epithelium at day 6 post-transfer. This proportion of hsCD8eff cells was comparable to that of the wild type hsCD8eff (FIG. 4A), indicating that CD103 expression is not required for initial migration of hsCD8eff into the intestinal epithelium. The numbers of wild type hsCD8eff cells, however, increased with time when compared to their CD103^{-/-} counterparts at later time points; i.e., the ratio of wild type: CD103^{-/-} hsCD8eff increased to 2:1 by day 21 and 4:1 by day 28. Selective retention of wild type vs. CD103^{-/-} hsCD8eff was also manifest in the spleen by day 28 post-transfer (FIG. 4A) but not in any of the non-lymphoid organs examined including the kidney, lung, and liver (FIG. 4B).

CD103 Expression Promotes CD8′ T-Cell Mediated Destruction Host Intestinal Epithelium—Hosts transferred with wild type 2C cells experienced more rapid mortality (MST=7.5±0.3 days, n=4) than those receiving CD103^{-/-}2C cells (MST=24.7±5.9 days, n=4) or 2C-DNR cells (MST=16.3±2.4 days, n=4) (data not shown). These data suggested that CD103 expression by hsCD8eff is causally related to the development of GVHD pathology and mortality.

To determine the impact of CD103 expression on GVHD mediated by polyclonal CD8⁺ T cells, two groups of BALB/c mice were lethally irradiated and adoptively transferred with CD8-enriched spleen cells (<0.5% CD4 contamination) obtained from either wild type or CD103^{-/-} donors (polyclonal B6 background) in combination with B6 BM. An additional control group received B6 BMC alone. Hosts from each group were monitored for survival, and intestinal specimens were analyzed histologically.

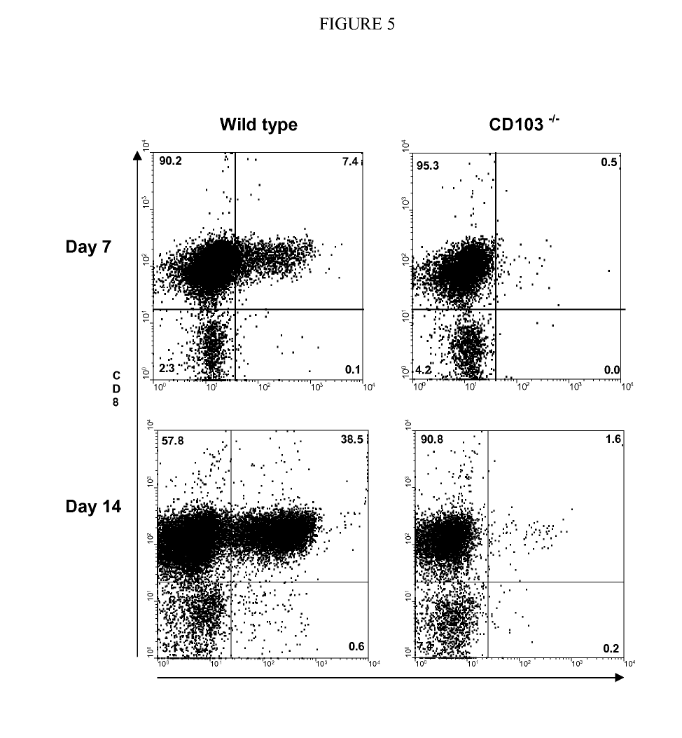

As shown in FIG. 5, FACS® analyses of donor-origin lymphocytes infiltrating the intestinal epithelium at day 14 post-BMT in recipients of wild type lymphocytes revealed a vast predominance of CD8 effectors (80-90% of infiltrating lymphocytes), ˜40% of which expressed CD103. Note that CD103 expression by gut-infiltrating lymphocytes was almost entirely confined to the CD8 subset (FIG. 5). There was a corresponding two-fold decrease in the abundance of CD103^{-/-} cells in the intestinal epithelium as compared to wild type counterparts (not shown). Thus, these data are consistent with a key role for CD103 in promoting both retention and pathogenic potential of polyclonal host-reactive CD8 effectors that infiltrate the host intestinal epithelium during GVHD.

### Example 3

### Separability of GVHD vs. GVT Effects Mediated By CD8⁺ T Cells

Bone marrow cells and purified CD8+ T cells from CD103−/− or wild type (WT) BALB/cJ (H-2d) mice were adoptively transferred into lethally-irradiated A/J (H-2a) mice together with the A/J fibroblast sarcoma (SaI/N). Incidence of GVHD and GVT was observed and target organs were assayed by histology and flow cytometry. Donors were primed with 1×107 A/J SC i.p. at day −5.

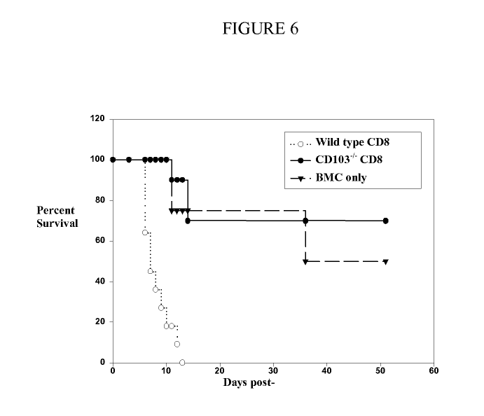

FIG. 6 shows that CD103−/− and wild-type CD8+ T cells both efficiently eliminated SaI/N tumors transplanted into syngeneic BALB/SCID mice with no tumor metastasis detected in either group at 10 weeks, thereby excluding the possibility that GVHD may contribute to tumor elimination. CD8+ T cells from wild-type (open circles) or CD103−/− (triangles) donors primed A/J alloantogens were adoptively transferred into histocompatible BALB/SCID mice together with the A/J fibrosarcoma SaI/N. A control group received SaI/N tumor only (closed circles). CD103−/− and WT CD8 T cells both efficiently eliminated SaI/N tumors transplanted into syngeneic BALB/scid mice, with no tumor metastasis detected in either group at 10 weeks. These data demonstrate that blocking CD103 activity in cells originating from the graft can abrogate GVHD while sparing beneficial GVT effects mediated by CD8+ T cells.

### Example 3

### Selective Depletion of CD8+ T cells in Bone Marrow

To test the ability to selectively destroy CD8+ T cells, we conjugated the M290 antibody with saporin to produce an anti-mouse CD103-immunotoxin (M290-SAP). The M290 is a rat IgG2a monoclonal antibody that blocks interaction of CD103 with its ligand, E-cadherin (Karecla, P. I., et al. Eur J Immunol 25:852-856 (1995); Roberts, K., and P. J. Kilshaw, Eur J Immunol 23:1630-1635 (1993), which are incorporated by reference). To date, however, the M290 antibody has not been shown to deplete CD103-expressing cells in vivo.

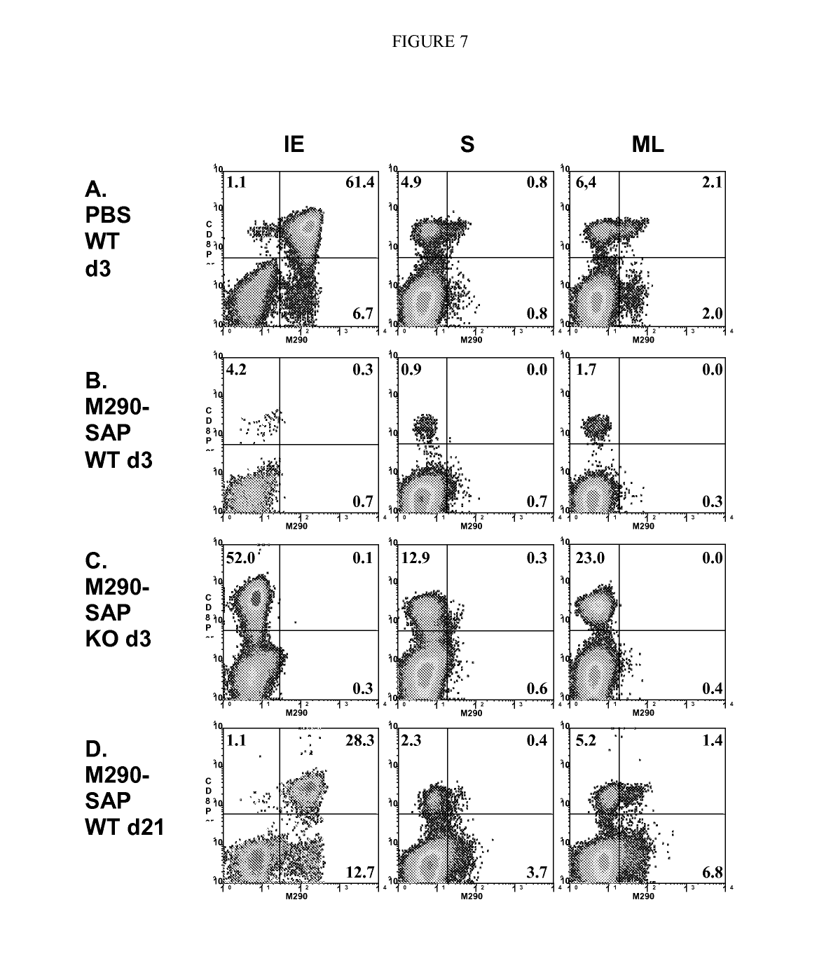

Wild-type C57BL/6 mice or CD103 knockout mice were injected i.p. with either PBS (0.5 ml) as control (A) or with 100 μg of the M290-MAP in 0.5 ml of PBS. After 3 days (A, B and C), cells were harvested from the intestinal epithelium (1E), spleen (S) or mesenteric lymph node (MN), and subsequently subjected to two-color FACS® using mAbs to CD8 and CD103. Within three days, post injection, CD8+ T cells in wild-type mice were depleted in all compartments examined, versus control mice, including the intestinal epithelium, spleen, and MN (FIG. 7B). In CD103 knockout mice, however, administration of M290-SAP into had no effect on total CD8+ cells (FIG. 7C). By 21 days post-treatment (FIG. 7D) the CD-103 expressing cells had partially recovered in each compartment examined.

### Example 4

### Inhibition of Allograft Rejection by M290-SAP-Mediated Depletion of CD130⁺CD8⁺ T cells

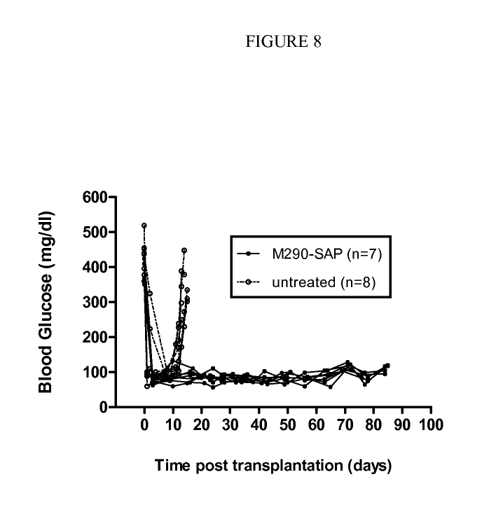

Importantly, further studies revealed that M290-SAP dramatically prolongs survival of transplanted tissues. As shown in FIG. 8, pancreatic islet allografts transplanted into normal mice were uniformly rejected within 2 weeks (blood glucose >200 mg/kg). This was not a technical failure because the islet transplants transiently restored normoglycemia to the diabetic recipients (FIG. 8). In contrast, islet allografts transplanted into recipients treated with 2 mg/kg i.p. of M290 at day 3 post-transplant (n=8) survived indefinitely (>80 days) (FIG. 8). In experiments not shown, recipients treated with the same dose of unconjugated M290 or isotype control (rat IgG) conjugated to saporin showed rejection times identical to those of untreated controls. In additional experiments, lower levels of M290 (0.5 and 1.0 mg/kg i.p.) also significantly prolonged graft survival but did not induce indefinite graft survival (data not shown). These data document the potential of depleting anti-CD103 mAb as a novel therapeutic strategy to prevent rejection of transplanted tissues and organs, and ameliorate graft-verses-host-disease pathology.

What is claimed is:

1. A method of selectively depleting CD103+ cells in vivo in a subject, said method comprising administering a composition to said subject, said composition comprising an anti-CD103 antibody conjugated to a lethal compound.

2. The method of claim 1, wherein said subject is a host subject receiving a graft.

3. The method of claim 2, wherein said graft is a bone marrow transplant.

4. A method of treating a subject for graft-versus-host disease (GVHD) comprising administering a composition to said subject, said composition comprising an anti-CD103 antibody conjugated to a lethal compound, wherein said composition is capable of selectively depleting CD103+ cells in vivo in said subject.

5. The method of claim 4, wherein said subject is a host subject receiving a graft.

6. The method of claim 5, wherein said graft is a bone marrow transplant.

7. The method of claim 6, wherein the GVHD is acute GVHD.

8. The method of claim 7, wherein the host subject is a human.

9. The method of claim 8, wherein the graft is an isograft in relation to the host subject.

10. The method of claim 8, wherein the graft is an allograft in relation to the host subject.

11. The method of claim 8, wherein the graft is a xenograft in relation to the host subject.

12. The method of claim 8, wherein said CD103+ cells are located at least in the intestine of said host subject.

13. The method of claim 8, wherein said CD103+ cells originating from the graft are CD8+ T cells.

14. The method of claim 8, wherein said composition is administered to said host subject prior to receiving said graft.

15. The method of claim 8, wherein said composition is administered to said host subject after receiving said graft.

16. The method of claim 15, wherein said lethal compound is selected from the group consisting of diphtheria toxin (DTA), shigella neurotoxin, botulinum toxin, tetanus toxin, cholera toxin, saporin, ricin and CSE-V2.

17. The method of claim 16, wherein said host subject is suffering from GVHD prior to administering said composition.

18. The method of claim 16, wherein said host subject is at risk of suffering from GVHD prior to administering said composition.

19. A composition comprising an anti-CD103 antibody conjugated to a lethal compound.

20. The composition of claim 19, wherein said lethal compound is selected from the group consisting of diphtheria toxin (DTA), shigella neurotoxin, botulinum toxin, tetanus toxin, cholera toxin, saporin, ricin and CSE-V2.


SEQUENCE LISTING (1 sequence)

SEQ ID NO: 1 - protein, 1179 aa, Homo sapiens
MWLFHTLLCIASLALLAAFNVDVARPWLTPKGGAPFVLSSLLHQDPSTNQTWLLVTSPRTKRTPGPLHRCSLVQDEILCHPVEHVPIPKGRHRGVTVVRSHHGVLICIQVLVRRPHSLSSELTGTCSLLGPDLRPQAQANFFDLENLLDPDARVDTGDCYSNKEGGGEDDVNTARQRRALEKEEEEDKEEEEDEEEEEAGTEIAIILDGSGSIDPPDFQRAKDFISNMMRNFYEKCFECNFALVQYGGVIQTEFDLRDSQDVMASLARVQNITQVGSVTKTASAMQHVLDSIFTSSHGSRRKASKVMVVLTDGGIFEDPLNLTTVINSPKMQGVERFAIGVGEEFKSARTARELNLIASDPDETHAFKVTNYMALDGLLSKLRYNIISMEGTVGDALHYQLAQIGFSAQILDERQVLLGAVGAFDWSGGALLYDTRSRRGRFLNQTAAAAADAEAAQYSYLGYAVAVLHKTCSLSYVAGAPQYKHHGAVFELQKEGREASFLPVLEGEQMGSYFGSELCPVDIDMDGSTDFLLVAAPFYHVHGEEGRVYVYRLSEQDGSFSLARILSGHPGFTNARFGFAMAAMGDLSQDKLTDVAIGAPLEGFGADDGASFGSVYIYNGHWDGLSASPSQRIRASTVAPGLQYFGMSMAGGFDISGDGLADITVGTLGQAVVFRSRPVVRLKVSMAFTPSALPIGFNGVVNVRLCFEISSVTTASESGLREALLNFTLDVDVGKQRRRLQCSDVRSCLGCLREWSSGSQLCEDLLLMPTEGELCEEDCFSNASVKVSYQLQTPEGQTDHPQPILDRYTEPFAIFQLPYEKACKNKLFCVAELQLATTVSQQELVVGLTKELTLNINLTNSGEDSYMTSMALNYPRNLQLKRMQKPPSPNIQCDDPQPVASVLIMNCRIGHPVLKRSSAHVSVVWQLEENAFPNRTADITVTVTNSNERRSLANETHTLQFRHGFVAVLSKPSIMYVNTGQGLSHHKEFLFHVHGENLFGAEYQLQICVPTKLRGLQVAAVKKLTRTQASTVCTWSQERACAYSSVQHVEEWHSVSCVIASDKENVTVAAEISWDHSEELLKDVTELQILGEISFNKSLYEGLNAENHRTKITVVFLKDEKYHSLPIIIKGSVGGLLVLIVILVILFKCGFFKRKYQQLNLESIRKAQLKSENLLEEEN